(19) United States
(12) Patent Application Publication
(10) Pub. No.: US 2026/0233019 A1
(43) Pub. Date: Aug. 13, 2026

(54) Magnetic Field Stimulation Device

(71) Applicant: National Central University, Taoyuan City (TW)

(72) Inventor: Po-Lei LEE, Taoyuan City (TW)

(21) Appl. No.: 19/213,622

(22) Filed: May 20, 2025

(30) Foreign Application Priority Data
Feb. 7, 2025 (TW) 114104483

(51) Int. Cl. A61N 2/00; A61N 2/02; G16H 40/67

(52) U.S. Cl. CPC A61N 2/004; A61N 2/02; G16H 40/67

(57) ABSTRACT

A magnetic field stimulation device is provided. The magnetic field stimulation device includes a wearable facial item, one or more coil films, and a control device. The one or more coil films are disposed on the wearable facial item. Each coil film includes a plurality of planar coils that are interconnected and a plurality of substrates that are stacked on top of each other, with the plurality of planar coils being respectively formed on the plurality of substrates. The control device is electrically connected to the one or more coil films. The control device generates a current driving signal according to a control parameter to drive the one or more coil films to generate magnetic fields. The current driving signal includes a plurality of waveforms, each of which has its own frequency and amplitude, such that the one or more coil films generate magnetic fields with corresponding characteristics according to these waveforms.

### CROSS REFERENCE TO RELATED APPLICATION

This application claims the priority of Taiwan Patent Application No. 114104483, filed on Feb. 7, 2025, entitled “Magnetic Field Stimulation Device,” and the disclosure of which is incorporated herein by reference.

### FIELD OF INVENTION

The present disclosure relates to a magnetic field stimulation device, and more particularly, to a magnetic field stimulation device that can be worn on the face.

### BACKGROUND OF INVENTION

Most of the facial stimulation devices currently on the market are masks that use LED light sources. These devices are primarily used for treatment, such as skin whitening, blemish reduction, and promoting collagen production. The penetration depth of LED light is typically affected by the wavelength of the light. For most LED lights, the penetration depth is generally between 1 mm and 6 mm. The skin consists of three layers: the epidermis, dermis, and subcutaneous tissue. The thickness of the epidermis is usually about 0.5 mm to 1.5 mm, the thickness of the dermis is about 1 mm to 4 mm, and the total thickness typically does not exceed 5 mm. Under these conditions, most of the energy from LED light is concentrated in the epidermis and dermis. Since the penetration depth of LED light mainly affects the epidermis and dermis, it is unable to effectively act on the deeper subcutaneous tissue, limiting its therapeutic effect on the subcutaneous tissue.

Because the subcutaneous tissue area is beyond the effective penetration depth of LED light, non-invasive treatments typically use radiofrequency (radio waves) or ultrasound energy to reach this area. For example, radio frequency skin lift and ultrasonic skin lifting are techniques that stimulate the subcutaneous tissue. Research shows that the current generated by radiofrequency skin lifting mainly conducts in the dermis and the collagen areas within the fibrous septa of the subcutaneous tissue.

Magnetic fields have the advantages of non-contact and strong penetration, making them an effective treatment option. Pulsed electromagnetic field (PEMF) technology, used for bone fracture healing, was approved by the U.S. FDA in 1797. In 2007, several pulsed electromagnetic field devices were FDA-approved and confirmed to have potential applications in treating depression, brain diseases, cellular collagen production, and skin tightening. However, PEMF devices are typically bulky and therefore cannot be worn on the face, limiting their application in the beauty field.

In recent years, lightweight devices using pulsed electromagnetic field technology have been gradually developed. Related patents include U.S. Patent US 20080058793 A1, U.S. Pat. No. 9,433,797 B2, and US2020353274 A1. However, the devices in these patents not only cannot be worn on the face for a long time, but also cannot generate sufficient magnetic field stimulation to achieve the desired therapeutic effect, thus limiting their practical application.

### SUMMARY OF INVENTION

In view of the shortcomings of the prior art mentioned above, the present disclosure proposes a magnetic field stimulation device.

The present disclosure provides a magnetic field stimulation device, comprising: a wearable facial item; one or more coil films, disposed on the wearable facial item, wherein each coil film includes a plurality of planar coils that are interconnected and a plurality of substrates that are stacked on top of each other, with the plurality of planar coils being respectively formed on the plurality of substrates; and a control device, electrically connected to the one or more coil films, wherein the control device generates a current driving signal based on a control parameter to drive the one or more coil films to generate magnetic fields; wherein the current driving signal includes a plurality of waveforms and each waveform has its own frequency and amplitude, such that the one or more coil films generate the magnetic fields with corresponding characteristics based on the waveforms.

In one embodiment of the present disclosure, the control device further includes a wireless transceiver, a microcontroller, a waveform generator, and a current amplifier.

In one embodiment of the present disclosure, the wireless transceiver is used to wirelessly receive the control parameter, and the microcontroller is used to receive the control parameter via wired means and/or receive the control parameter from the wireless transceiver, wherein the microcontroller generates a control signal based on the control parameter.

In one embodiment of the present disclosure, the waveform generator generates one of an AM signal, a PWM signal, or a CHIRP signal based on the control signal, and the current amplifier generates the current driving signal based on one of the AM signal, the PWM signal, or the CHIRP signal.

In one embodiment of the present disclosure, the AM signal is associated with a basic wave and a carrier wave.

In one embodiment of the present disclosure, the CHIRP signal is associated with an initial fundamental frequency, a terminal frequency, and an amplification rate.

In one embodiment of the present disclosure, the wearable facial item includes a mask substrate and ear hook elements disposed on both sides of the mask substrate, wherein the one or more coil films are disposed on the mask substrate, and the control device is disposed on either the mask substrate or the ear hook elements.

In one embodiment of the present disclosure, the wearable facial item includes a glasses substrate and ear hook elements disposed on both sides of the glasses substrate, wherein the one or more coil films are disposed on the glasses substrate, and the control device is disposed on the ear hook elements.

In one embodiment of the present disclosure, further comprising a power source to supply power to the control device.

### DESCRIPTION OF DRAWINGS

FIG. 1 shows a schematic diagram of a magnetic field stimulation device of the present disclosure.

FIG. 2 shows a schematic diagram of a coil film and the magnetic field B generated by it.

FIG. 3 shows a side view schematic diagram of the coil film of the magnetic field stimulation device of the present disclosure.

FIG. 4 shows a schematic diagram of the connection of a plurality of planar coils in the coil film.

FIG. 5 shows a schematic diagram of the first embodiment of the magnetic field stimulation device of the present disclosure.

FIG. 6 shows a schematic diagram of the second embodiment of the magnetic field stimulation device of the present disclosure.

FIG. 7 shows a schematic diagram of the third embodiment of the magnetic field stimulation device of the present disclosure.

FIG. 8 shows a schematic diagram of the fourth embodiment of the magnetic field stimulation device of the present disclosure.

FIG. 9 shows a schematic diagram of the fifth embodiment of the magnetic field stimulation device of the present disclosure.

FIG. 10 shows a schematic diagram of the sixth embodiment of the magnetic field stimulation device of the present disclosure.

### DETAILED DESCRIPTION OF PREFERRED EMBODIMENTS

In order to make the above and other objectives, features, and advantages of the present disclosure more obvious and understandable, the following exemplifies the preferred embodiments of the present disclosure, combined with the accompanying drawings, and describe in detail as follows.

The drawings in this application are schematic. Specifically, the proportions, sizes, or appearances of the components in the drawings are presented in a schematic manner and do not represent the actual proportions, sizes, or appearances of the components. For example, the lengths, widths, and proportions of the mask substrate, glasses substrate, and coil film(s) (including the coil and substrate) in the present disclosure are not shown according to the actual product dimensions. Additionally, for simplicity, the plurality of planar coils and coil films hereafter may, in some cases, be represented by a single planar coil in the drawings.

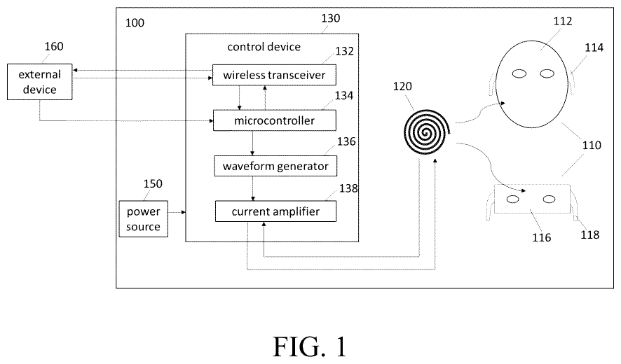

Firstly, refer to FIG. 1. FIG. 1 shows a schematic diagram of a magnetic field stimulation device of the present disclosure. The magnetic field stimulation device 100 includes a wearable facial item 110, one or more coil films 120, a control device 130, and a power source 150. In one embodiment, the wearable facial item 110 can be a mask. In this embodiment, the mask can include only the mask substrate 112, or it can include the mask substrate 112 and mask ear hook elements 114 arranged on both sides of the mask substrate 112. The coil film(s) 120 is/are disposed on the mask substrate 112. The control device 130 can be disposed on the mask substrate 112 or on the mask ear hook element 114. In another embodiment, the wearable facial item 110 can be glasses, which include a glasses substrate 116 and glasses ear hook elements 118 arranged on both sides of the glasses substrate 116. In this embodiment, the coil film(s) 120 is/are disposed on the glasses substrate 116, and the control device 130 is disposed on the glasses ear hook element 118.

In various embodiments, the mask substrate 112 can be oval, circular, or rectangular, but is not limited thereto. The glasses substrate 116 can be rectangular or oval, but is not limited thereto. Depending on the appearance of the wearable facial item and the user's needs, the coil film(s) 120 can be placed on the surface of the wearable facial item 110 or embedded within it. Additionally, the number, size, and shape of the coil film(s) 120 can be adjusted. For example, a large number of small coil films 120 can be arranged on the wearable facial item 110, or a few large coil films 120 can be arranged on the wearable facial item 110. The appearance of the coil film(s) 120 and its/their planar coils can be designed to be rectangular, circular, and/or oval. The structure and function of the coil film(s) 120 will be described later.

In various embodiments, the control device 130 is electrically connected to the coil film(s) 120. The control device 130 generates a current drive signal based on a control parameter/control parameters to drive the coil film(s) 120 to generate a magnetic field/magnetic fields. The current drive signal can include a plurality of waveforms, with each waveform having its own frequency and amplitude, allowing the coil film(s) 120 to generate corresponding magnetic field characteristics based on these waveforms. The power source 150 provides power to the control device 130. The power source 150 can include a battery or a mobile power source, but is not limited thereto. The control device 130 can receive control parameters from an external device 160. The external device 160 can include any device capable of communicating with the control device 130 and various input devices, such as a personal computer, laptop, tablet, smartphone, keyboard, and touch panel, etc.

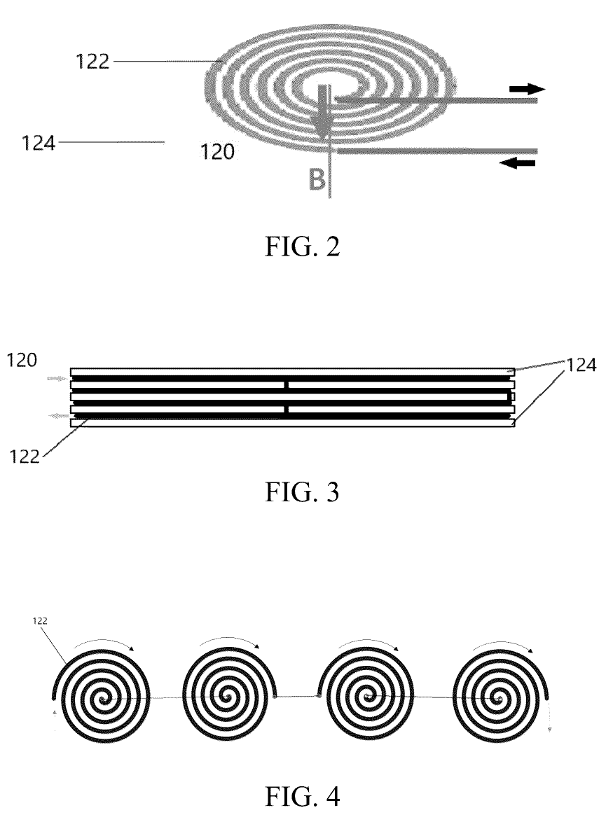

Please refer to FIG. 2. FIG. 2 shows a schematic diagram of a coil film and the magnetic field B generated by it. In one embodiment, as shown in FIG. 2, the coil film 120 is composed of a single planar coil 122 and a single substrate 124, where the planar coil 122 is formed on the substrate 124. When current passes through the planar coil 122, its central region generates the magnetic field B.

Please refer to FIGS. 3 and 4. FIG. 3 shows a side view schematic diagram of the coil film of the magnetic field stimulation device of the present disclosure. FIG. 4 shows a schematic diagram of the connection of a plurality of planar coils in the coil film. In another embodiment, as shown in FIG. 3, the coil film 120 is composed of four planar coils 122 and four substrates 124. In FIG. 3, each planar coil 122 is respectively formed on a substrate 124, and the planar coils 122 are connected to each other, while the substrates 124 are stacked on top of each other. In other words, the planar coils 122 are also stacked on top of each other. It is noteworthy that these planar coils 122 are stacked according to a common central axis, meaning the central axes of each planar coil 122 overlap. For clarity, as shown in FIG. 4, the four planar coils 122 are expended, from left to right, labeled as the first to the fourth coil. The outer end of the first coil is connected to the current signal input, and the central endpoint of the first coil is connected to the central endpoint of the second coil. The outer end of the second coil is connected to the outer end of the third coil. The central endpoint of the third coil is connected to the central endpoint of the fourth coil, and the outer end of the fourth coil is connected to the current signal output. The arrows in FIG. 4 represent the direction of current flow. According to the description in FIG. 2, the central region of a single planar coil 122 generates a magnetic field B. When the coil film 120 is made up of multiple stacked and connected planar coils 122, due to the consistent direction of current in each planar coil 122, the central region of each planar coil 122 synchronously generates a magnetic field B in the same direction. The collective magnetic fields B will form a stronger overall magnetic field.

Returning to FIG. 1, in one embodiment, the control device 130 further includes a wireless transceiver 132, a microcontroller 134, a waveform generator 136, and a current amplifier 138. The wireless transceiver 132 is used to wirelessly receive control parameters, and the microcontroller 134 is used to receive control parameters wired and/or receive control parameters from the wireless transceiver 132. The microcontroller 134 can generate control signals based on the control parameters. The waveform generator 136 can generate one of an AM signal, PWM signal, or CHIRP signal based on the control signal. The current amplifier 138 can generate a current drive signal based on one of the AM signal, PWM signal, or CHIRP signal. As mentioned earlier, the current drive signal is used to drive the coil film(s) 120 to generate a magnetic field/magnetic fields. The amplitude, frequency, and waveform of the current drive signal (amplified AM signal, PWM signal, and CHIRP signal) will collectively determine the magnetic field characteristics, such as strength, frequency, and penetration depth.

In one embodiment, an AM signal is generated by modulating with a 50 Hz basic wave and a 30 kHz carrier wave. Additionally, a CHIRP signal is generated by modulation with an initial fundamental frequency, a terminal frequency, and an amplification rate.

In an embodiment of the present disclosure, the CHIRP signal can be determined by the following formula:

$$x ( t ) = a * [ t + t cos ( 2 π ( {f}_{0} t + \frac{1}{2} {kt}^{2} ) ) ] ∘$$

Where:
a represents the amplification rate;
t=0~T, where T represents the period of one CHIRP signal, for example, if each wave has T=30 ms, then its frequency is 33 Hz;
fo is the initial fundamental frequency of each wave, for example, 30 Hz;
fl is the terminal frequency of each wave, for example, 3.3 kHz;

$$k = ( f 1 - f 0 ) / T .$$

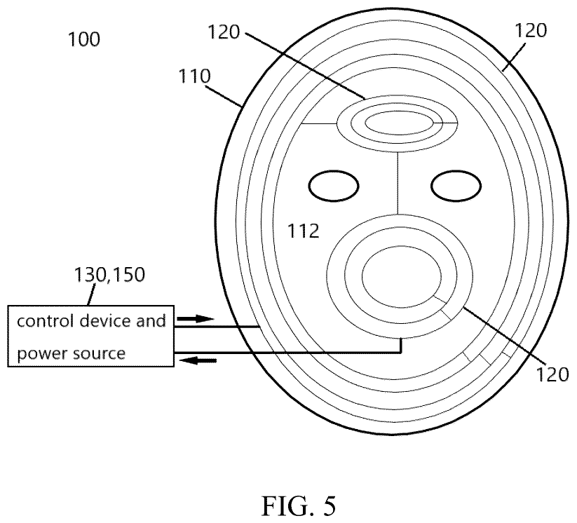

Please refer to FIG. 5, FIG. 5 shows a schematic diagram of the first embodiment of the magnetic field stimulation device of the present disclosure. In the embodiment of FIG. 5, the wearable facial item 110 of the magnetic field stimulation device 100 is implemented as a mask, which has only an oval-shaped mask substrate 112. Several oval-shaped coil films 120 are distributed on the mask substrate 112 according to its shape. In this embodiment, one large coil film 120 is arranged in the surrounding area of the mask substrate 112, and two medium-sized coil films 120 are arranged in the central area of the mask substrate 112. In this embodiment, the control device 130 and power supply 150 are implemented as not being configured on the wearable facial item 110, with the control device 130 connected to the coil films 120 only through wires.

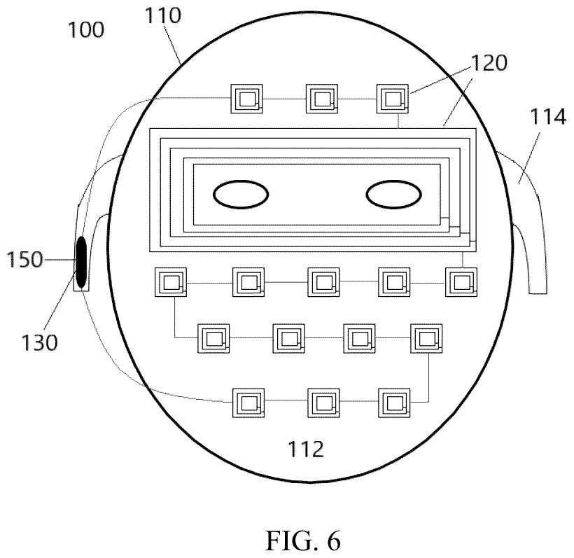

Please refer to FIG. 6. FIG. 6 shows a schematic diagram of the second embodiment of the magnetic field stimulation device of the present disclosure. In the embodiment of FIG. 6, the wearable facial item 110 of the magnetic field stimulation device 100 is implemented as a mask, which includes an oval-shaped mask substrate 112 and mask ear-hook elements 114. Multiple rectangular coil films 120 are distributed on the mask substrate 112. In this embodiment, one large coil film 120 is arranged around the eyes area of the mask substrate 112, and several small coil films 120 are arranged in the other areas of the mask substrate 112. The control device 130 and power supply 150 are implemented as being disposed on the mask ear-hook elements 114.

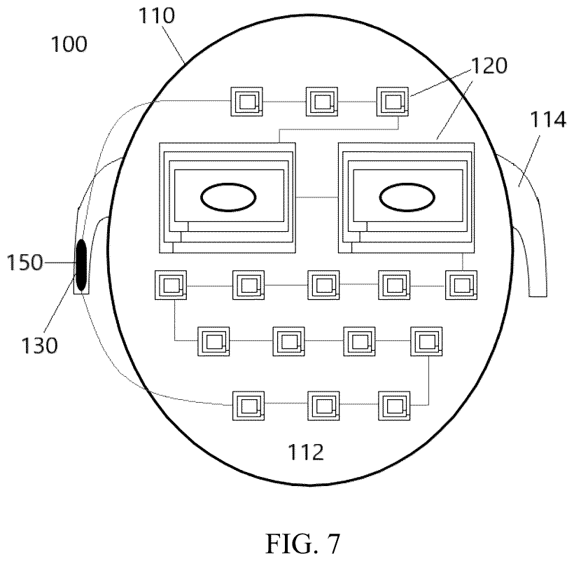

Please refer to FIG. 7. FIG. 7 shows a schematic diagram of the third embodiment of the magnetic field stimulation device of the present disclosure. In the embodiment of FIG. 7, the wearable facial item 110 of the magnetic field stimulation device 100 is implemented as a mask, which includes an oval-shaped mask substrate 112 and mask ear-hook elements 114. Multiple rectangular coil films 120 are distributed on the mask substrate 112. In this embodiment, two medium-sized coil films 120 are arranged around the eye areas of the mask substrate 112, and several small coil films 120 are arranged in the other areas of the mask substrate 112. The control device 130 and power supply 150 are implemented as being disposed on the mask ear-hook elements 114.

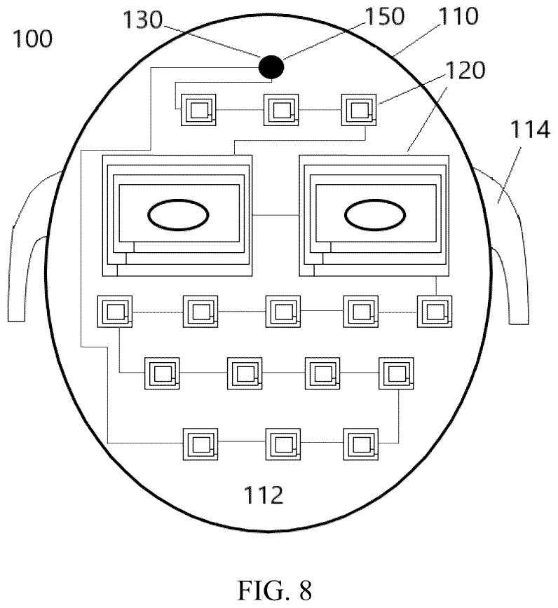

Please refer to FIG. 8. FIG. 8 shows a schematic diagram of the fourth embodiment of the magnetic field stimulation device of the present disclosure. In the embodiment of FIG. 8, the wearable facial item 110 of the magnetic field stimulation device 100 is implemented as a mask, which includes an oval-shaped mask substrate 112 and mask ear-hook elements 114. Multiple rectangular coil films 120 are distributed on the mask substrate 112. In this embodiment, two medium-sized coil films 120 are arranged around the eye areas of the mask substrate 112, and several small coil films 120 are arranged in the other areas of the mask substrate 112. The difference from the embodiment in FIG. 7 is that the control device 130 and power supply 150 are implemented as being disposed on the mask substrate 112.

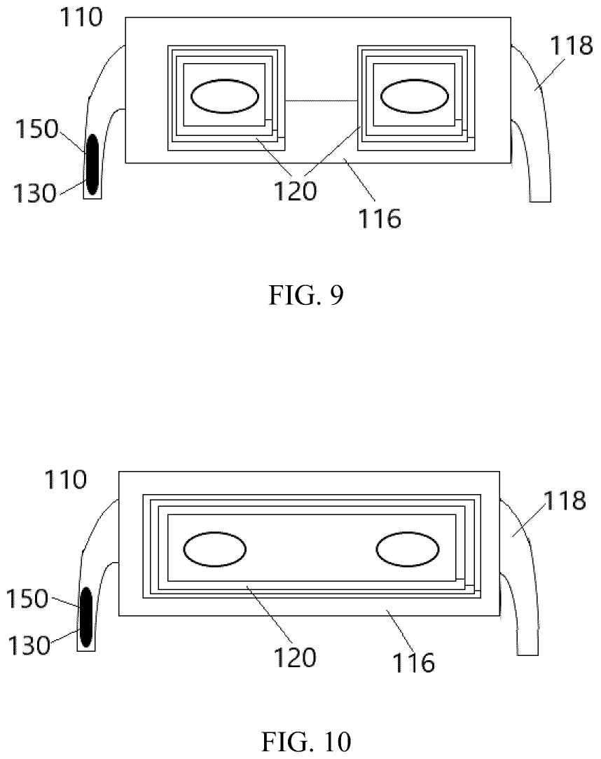

Please refer to FIG. 9. FIG. 9 shows a schematic diagram of the fifth embodiment of the magnetic field stimulation device of the present disclosure. In the embodiment of FIG. 9, the wearable facial item 110 of the magnetic field stimulation device 100 is implemented as glasses, which include a rectangular glasses substrate 116 and glasses ear-hook elements 118. Two rectangular coil films 120 are disposed on the glasses substrate 116, surrounding the areas around both eyes. In this embodiment, the control device 130 and power supply 150 are implemented as being disposed on the glasses ear-hook elements 118.

Please refer to FIG. 10. FIG. 10 shows a schematic diagram of the sixth embodiment of the magnetic field stimulation device of the present disclosure. In the embodiment of FIG. 10, the wearable facial item 110 of the magnetic field stimulation device 100 is implemented as glasses, which include a rectangular glasses substrate 116 and glasses ear-hook elements 118. A rectangular coil film 120 is disposed on the glasses substrate 116, specifically surrounding the areas around both eyes. In this embodiment, the control device 130 and power supply 150 are implemented as being disposed on the glasses ear-hook elements 118.

Compared to the conventional technologies, the present disclosure has the following significant advantages: The magnetic field stimulation device of the present disclosure has high flexibility and adjustability, allowing parameters to be adjusted based on different treatment needs, thus achieving targeted and personalized therapeutic effects. Specifically, this device can select or adjust parameters based on the specific conditions of the treatment target to generate several current waveforms with different characteristics. Each current waveform generates corresponding magnetic field characteristics. These magnetic field characteristics can produce optimized therapeutic effects for different biological tissues or conditions.

For example, for superficial tissues or surface layers (such as skin, nerves, or superficial muscles), a magnetic field waveform with higher amplitude and high-frequency pulses can promote a strong response in the nerves or muscles, helping to stimulate more noticeable therapeutic effects. In contrast, if the treatment target is deeper tissues (such as deep muscles, bones, or internal organs), magnetic field waveforms with lower frequency and larger pulse width can be used. Such magnetic field waveforms can more effectively penetrate the superficial tissues and concentrate the magnetic field energy on deeper target areas, thus achieving the desired therapeutic effects.

The above is only exemplary, rather than restrictive. Any equivalent modifications or changes without departing from the spirit and scope of the present disclosure should fall within the scope of the appended claims.

What is claimed is:

1. A magnetic field stimulation device, comprising:
a wearable facial item;
one or more coil films, disposed on the wearable facial item, wherein each coil film includes a plurality of planar coils that are interconnected and a plurality of substrates that are stacked on top of each other, with the plurality of planar coils being respectively formed on the plurality of substrates; and
a control device, electrically connected to the one or more coil films, wherein the control device generates a current driving signal based on a control parameter to drive the one or more coil films to generate magnetic fields;
wherein the current driving signal includes a plurality of waveforms and each waveform has its own frequency and amplitude, such that the one or more coil films generate the magnetic fields with corresponding characteristics based on the waveforms.

2. The device according to claim 1, wherein the control device further includes a wireless transceiver, a microcontroller, a waveform generator, and a current amplifier.

3. The device according to claim 2, wherein the wireless transceiver is used to wirelessly receive the control parameter, and the microcontroller is used to receive the control parameter via wired means and/or receive the control parameter from the wireless transceiver, wherein the microcontroller generates a control signal based on the control parameter.

4. The device according to claim 3, wherein the waveform generator generates one of an AM signal, a PWM signal, or a CHIRP signal based on the control signal, and the current amplifier generates the current driving signal based on one of the AM signal, the PWM signal, or the CHIRP signal.

5. The device according to claim 4, wherein the AM signal is associated with a basic wave and a carrier wave.

6. The device according to claim 4, wherein the CHIRP signal is associated with an initial fundamental frequency, a terminal frequency, and an amplification rate.

7. The device according to claim 1, wherein the wearable facial item includes a mask substrate and ear hook elements disposed on both sides of the mask substrate, wherein the one or more coil films are disposed on the mask substrate, and the control device is disposed on either the mask substrate or the ear hook elements.

8. The device according to claim 1, wherein the wearable facial item includes a glasses substrate and ear hook elements disposed on both sides of the glasses substrate, wherein the one or more coil films are disposed on the glasses substrate, and the control device is disposed on the ear hook elements.

9. The device according to claim 1, further comprising a power source to supply power to the control device.
